(19) United States
(12) Patent Application Publication
(10) Pub. No.: US 2005/0131415 A1
(43) Pub. Date: Jun. 16, 2005

(54) Adaptive apparatus for driving a threaded device into material such as a biological tissue

(76) Inventors: Trevor C. Hearn, Belair (AU); Karen J. Reynolds, Bellevue Heights (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/972,076

(22) Filed: Oct. 21, 2004

Related U.S. Application Data
(63) Continuation of application No. PCT/AU03/00499, filed on Apr. 24, 2003.

(30) Foreign Application Priority Data
Apr. 24, 2002 (AU) PS1951/02

(51) Int. Cl. A61B 17/32

(52) U.S. Cl. 606/80

(57) ABSTRACT

An adaptive apparatus for driving a threaded device into material, the apparatus including a rotating driving bit, a sensor for sensing during said driving a first quantity related to the material's properties, memory means for storing sensed values of said first quantity, and a feedback arrangement which processes the values sensed by the sensor in comparison to known data to characterise the material and to determine a shut-off condition at which safe and effective engagement of the threaded device in the material is achieved, wherein the feedback arrangement ceases rotation of said driving bit when said shut-off condition is achieved. In another form of the invention, there is provided a method of driving a threaded device into material, said method including the steps of: driving the threaded device into said material by way of a rotating driving bit; sensing during said driving a first quantity related to material properties of the material; storing sensed values of said first quantity; processing the values sensed by the sensor in comparison to known data to characterise the material and to determine a shut-off condition at which safe and effective engagement of the threaded device in the material is achieved; and ceasing rotation of said driving bit when said shut-off condition is achieved.

### CROSS REFERENCE TO RELATED APPLICATIONS

This application is a continuation of and claims priority to PCT Application PCT/AU03/00499, filed on Apr. 24, 2003 and entitled Adaptive Apparatus for Driving a Threaded Device into Material such as Biological Tissue.

### FIELD OF THE INVENTION

This invention relates to a feedback-controlled driver, and particularly, but not exclusively, to a feedback-controlled rotary handpiece for anchoring threaded devices in material with variable properties.

### BACKGROUND OF THE INVENTION

Numerous mechanical procedures require the drilling of material and anchoring of threaded devices. The resulting structural integrity largely depends on the torque attained in the threaded device. The screw torque can greatly affect the mechanical behaviour of complex constructs. Over-tightening, however, can result in failure of the device itself, or in host material failure around the thread, and complete loss of mechanical function. Torque failure levels for manufactured devices are predictable, however, torque levels corresponding to host material failure vary greatly, due to the wide variation in properties between materials in general, within biological materials in particular and between locations within a material.

Threaded implants are common in dental surgery for anchoring implants in the mandible or teeth. They are also common in Orthopaedic Surgery, where there are many applications in reconstructive procedures and injury repairs.

In particular, one of the most common surgical procedures in orthopaedics is the internal fixation of fractures using a threaded device in the form of a bone screw. Bone screws are an important mechanical device used to stabilise and align bone fragments, or fix a bone plate to bone. During insertion, torque is applied to the screw so as to advance it into the bone until the head strikes the bone cortex or bone plate. Further torque is converted into axial tension along the length of the screw, and compression between the fragments.

Over-tightening occurs when the compressive force between the head and the bone or bone plate results in failure of the threaded device or damage to the bone around the threaded device. Both scenarios result in the loss of fracture fixation and stability. Over-tightening is an important issue because the optimal tightening a surgeon applies to a bone screw is an acquired skill, an intuitive feeling which is developed over years of training and experience. The optimal torque which is applied depends on the patient's overall health, lifestyle and age but most importantly their bone density and strength which varies greatly between individuals.

One previously proposed apparatus for drilling bone and driving a screw into the bone comprises a drill and a screwdriver which is operated manually and requires that the user determine head contact by the “feel” of the screwdriver during the driving operation, and by observing the bone and the threaded device. Such an apparatus requires great skill of the user in order to correctly “feel” the optimal torque, and is subject to over-tightening and thread-stripping of the host material, especially when used by inexperienced users. Another type of previously proposed apparatus for tightening threaded devices into bone material is disclosed in U.S. Pat. No. 4,359,906. The apparatus includes a torque-limiting driver which requires a predetermined torque limit to be input to the apparatus so that it can be shut off when the predetermined torque limit is achieved. However, any such torque or rotational angle-limiting apparatus which requires a predetermined setting is of limited use as the optimal torque (and optimal rotational angle of a threaded device) will vary greatly from case to case depending on such factors as quality of the host material, and quality and geometry of the threaded device. In another previously proposed apparatus, disclosed in International Patent Application No. PCT/SE97/02096, a driver for driving a threaded device into bone is connected to an external display which provides additional feedback to the operator, but such an apparatus is limited by the constraints of connecting cables between the driver and the display, and operator attention.

### SUMMARY OF THE INVENTION

In accordance with one aspect of the invention, there is provided an adaptive apparatus for driving a threaded device into material, the apparatus including a rotating driving bit, a sensor for sensing during said driving a first quantity related to the material's properties, memory means for storing sensed values of said first quantity, and a feedback arrangement which processes the values sensed by the sensor in comparison to known data to characterise the material and to determine a shut-off condition at which safe and effective engagement of the threaded device in the material is achieved, wherein the feedback arrangement ceases rotation of said driving bit when said shut-off condition is achieved.

Preferably, the processing of the values sensed by the sensor is performed continuously.

Preferably, contact between a head of the threaded device and the material is automatically detected by the apparatus, and initial values of said first quantity are sensed prior to said contact. More preferably, said contact between the head of the threaded device and the material is detected by sensing said first quantity.

Preferably, the apparatus includes a further sensor for sensing a second quantity, and subsequent values of said second quantity are stored in said memory means and are processed in combination with values of said first quantity and in comparison to known data to characterise the material and to determine said shut-off condition.

Preferably, the apparatus includes a drill bit for drilling the material so that said threaded device can be driven into a hole formed by said drill bit.

Preferably, the first-mentioned sensor senses said first quantity during said drilling to supplement values of said first quantity sensed during driving, and subsequent values of said first quantity sensed during said drilling are stored in said storage means during said drilling, and said feedback arrangement processes these values in comparison to known data to characterise the material and to determine said shut-off condition at which safe and effective engagement of the threaded device in the material is achieved.

Preferably, the rotating driving bit is powered by a motor, and the feedback arrangement processes the quantity or quantities sensed by the one or more sensors to control rotation of the motor to prevent over-tightening.

Preferably, said first quantity is or is directly related to an amount of resistive torque exerted by the material on the threaded device.

In an alternative embodiment, the rotating driving bit is driven pneumatically, said first quantity is or is related to pressure, and control is effected by means of a regulator.

Preferably, the second quantity is angular rotation of either the drilling or driving bit. Alternatively, the first quantity may be axial force on the drilling or driving bit. Alternatively, the second quantity may be axial displacement of the threaded device in the material.

Preferably, the apparatus is self-contained and is portable in a hand-held form.

Preferably, the threaded device is a screw.

Preferably, the feedback arrangement characterises the material by comparing the magnitude of values of said first quantity, sensed prior to contact of the head of the threaded device on the surface of the material, to known data for different material types to isolate a relevant set of known data, and the shut-off condition is determined by ascertaining an expected failure threshold from the relevant set of known data and by applying a safety factor to the expected failure threshold.

In accordance with another aspect of the invention, there is provided a method of driving a threaded device into material, said method including the steps of:
driving the threaded device into said material by way of a rotating driving bit;
sensing during said driving a first quantity related to material properties of the material;
storing sensed values of said first quantity;
processing the values sensed by the sensor in comparison to known data to characterise the material and to determine a shut-off condition at which safe and effective engagement of the threaded device in the material is achieved; and
ceasing rotation of said driving bit when said shut-off condition is achieved.

Preferably, the processing step is performed continuously.

Preferably, said method further includes the steps of automatically detecting contact between a head of the threaded device and the surface of the material, and sensing initial values of said first quantity prior to said contact. More preferably, said method includes the step of automatically detecting contact between the head of the threaded device and the material by sensing said first quantity.

Preferably, said method further includes the steps of sensing a second quantity, storing subsequent values of said second quantity, and processing values of said second quantity in combination with values of said first quantity and in comparison to known data to characterise the material and to determine said shut-off condition.

Preferably, said method further includes the step of drilling said material so that said threaded device can be driven into a hole formed by said drilling step.

Preferably, said method includes the steps of sensing subsequent values of said first quantity during said drilling to supplement values of said first quantity sensed during driving, storing these sensed values, and processing the values sensed during the drilling step in comparison to known data to characterise the material and to determine said shut-off condition at which safe and effective engagement of the threaded device in the material is achieved.

Preferably, said first quantity is or is directly related to an amount of resistive torque exerted by the material on the threaded device. In one embodiment, said first quantity is the current of a motor operating the driving bit.

In an alternative embodiment, the rotating driving bit is driven pneumatically, said first quantity is or is related to pressure, and control is effected by means of a regulator.

Preferably, the second quantity is angular rotation of the driving bit.

Alternatively, the first quantity may be axial force exerted by the material on the driving bit.

Alternatively, the second quantity may be axial displacement of the threaded device in the material.

Preferably, the method further includes the steps of characterising the material by comparing the magnitude of values of said first quantity sensed prior to contact of the head of the threaded device on the surface of the material to known data for different material types to isolate a relevant set of known data, and determining the shut-off condition by ascertaining an expected failure threshold from the relevant set of known data and by applying a safety factor to the expected failure threshold.

Preferably, the material is bone. In one alternative embodiment, the material is wood. In another alternative embodiment, the material is synthetic (non-biological). In yet a further alternative embodiment, the material is tooth material.

In accordance with another aspect of the invention, there is provided an adaptive apparatus for driving a threaded device relative to a body of material, the apparatus including a rotating driving bit, a sensor for sensing a first quantity related to a material property of the body, memory means for storing sensed values of said first quantity, and a feedback arrangement which processes the values sensed by the sensor in comparison to known data to characterise the material of the body and to determine a shut-off condition at which safe and effective engagement of the threaded device to the material is achieved, wherein the feedback arrangement ceases rotation of said driving bit when said shut-off condition is achieved.

In accordance with another aspect of the invention, there is provided a method of driving a threaded device relative to a body of material, said method including the steps of:
driving the threaded device relative to said material by way of a rotating driving bit;
sensing a first quantity related to a material property of the body;
storing sensed values of said first quantity;
processing the values sensed by the sensor in comparison to known data to characterise the material of the body and to determine a shut-off condition at which safe and effective engagement of the threaded device to the material is achieved; and
ceasing rotation of said driving bit when said shut-off condition is achieved.

### BRIEF DESCRIPTION OF THE DRAWINGS

The invention will now be described, by way of example only, with reference to the accompanying drawings, in which:

FIG. 1 is a flow chart illustrating the basic components of an apparatus for driving a threaded device in accordance with the present invention;

FIG. 2 illustrates the type of signals to be detected by the apparatus of FIG. 1;

FIG. 3 is a diagrammatic side view of a portable drilling and driving handpiece of the apparatus of FIG. 1, showing the arrangement of the internal components;

FIG. 4 is a diagrammatic side view of the handpiece of FIG. 3 in operation;

FIG. 5 is a circuit diagram used in the apparatus of FIGS. 3 and 4;

FIG. 6 is a graph of three example plots of Torque versus Time in tightening a 7.0 mm screw to failure in polyurethane foam of density 0.3 μm/cc;

FIG. 7 is a graph of Torque versus Time in tightening a 7.0 mm screw in polyurethane foam of density 0.3 gm/cc, from a laboratory test of an adaptive tightening method in accordance with the present invention;

FIG. 8 is a graph of three example plots of Current versus Time in tightening a 7.0 mm screw to failure in polyurethane foam of density 0.3 gm/cc;

FIG. 9 is a graph of Current versus Time in tightening a 7.0 mm screw in polyurethane foam of density 0.3 gm/cc, from a laboratory test of an adaptive tightening method in accordance with the present invention;

FIG. 10 is a graph of three example plots of Torque versus Time in tightening a 7.0 mm screw to failure in polyurethane foam of density 0.2 gm/cc;

FIG. 11 is a graph of Torque versus Time in tightening a 7.0 mm screw in polyurethane foam of density 0.2 gm/cc, from a laboratory test of an adaptive tightening method in accordance with the present invention;

FIG. 12 is a graph of three example plots of Current versus Time in tightening a 7.0 mm screw to failure in polyurethane foam of density 0.2 gm/cc;

FIG. 13 is a graph of Current versus Time in tightening a 7.0 mm screw in polyurethane foam of density 0.2 gm/cc, from a laboratory test of an adaptive tightening method in accordance with the present invention;

FIG. 14 is a graph of two example plots of Torque versus Time in tightening a bone screw to failure in cancellous bone;

FIG. 15 is a graph of Torque versus Time in tightening a bone screw in cancellous bone, from a laboratory test of an adaptive tightening method in accordance with the present invention;

FIG. 16 is a graph of two example plots of Current versus Time in tightening bone screw to failure in cancellous bone;

FIG. 17 is a graph of Current versus Time in tightening a bone screw in cancellous bone, from a laboratory test of an adaptive tightening method in accordance with the present invention; and

FIG. 18 contains two tables showing the results of further laboratory measurements testing an adaptive apparatus in accordance with the present invention in various host materials.

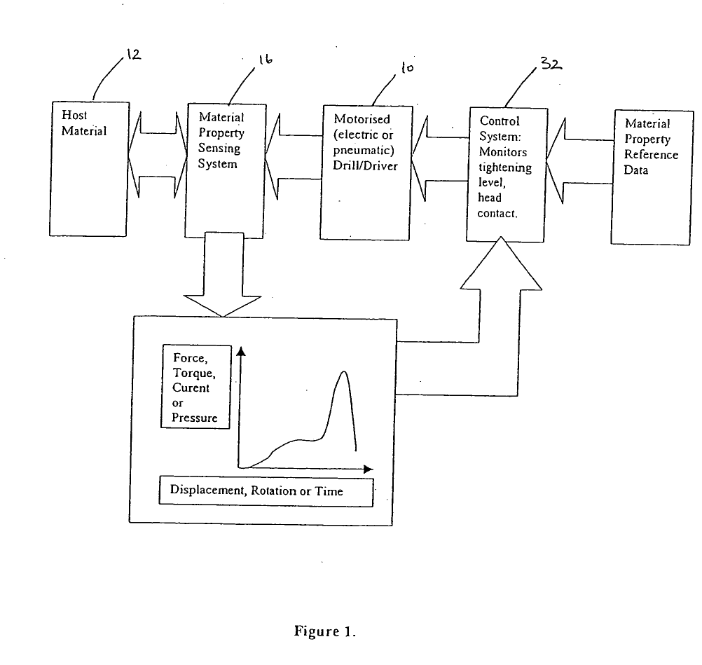

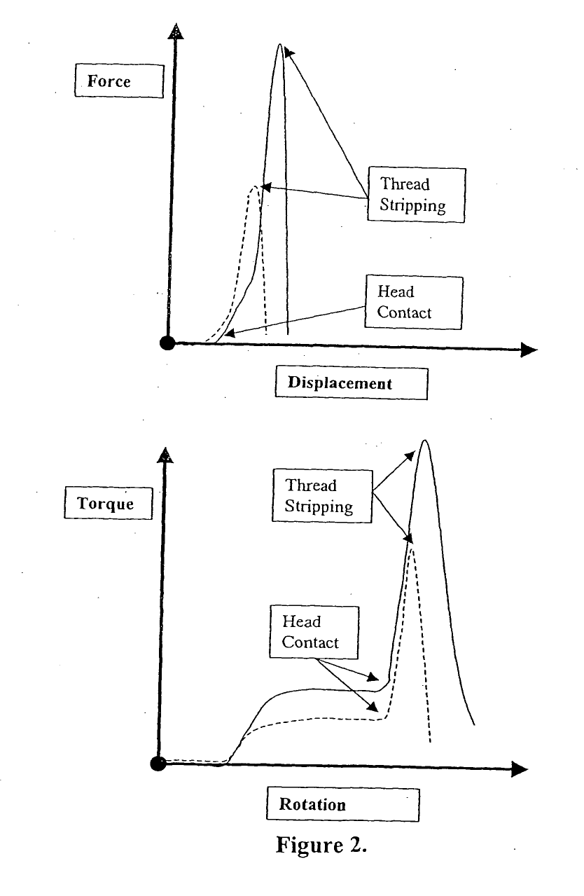

### DETAILED DESCRIPTION OF PREFERRED EMBODIMENTS

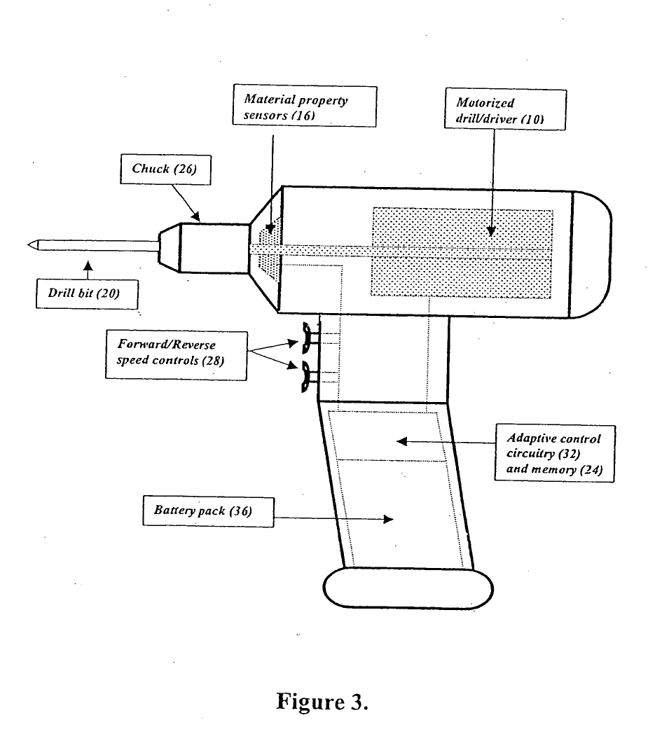

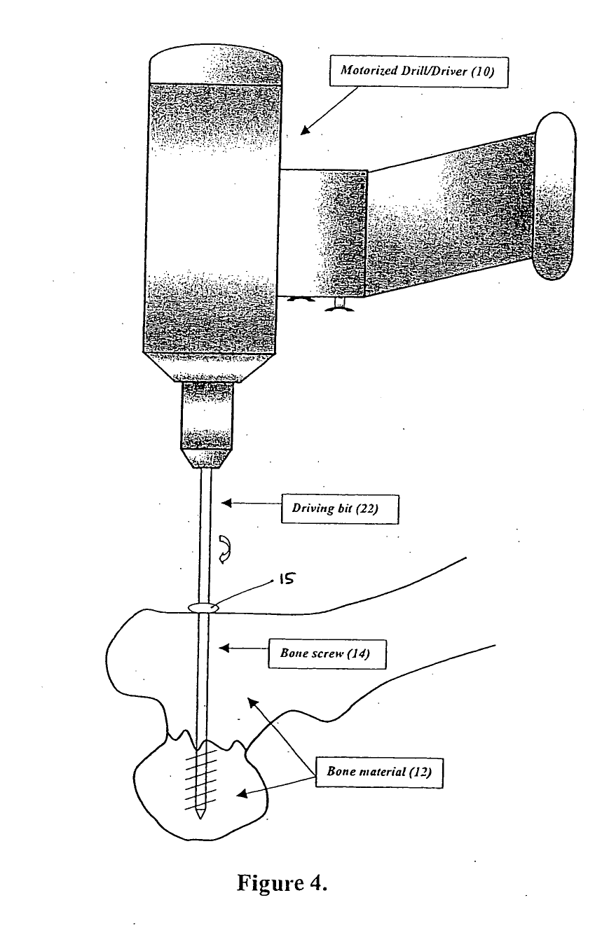

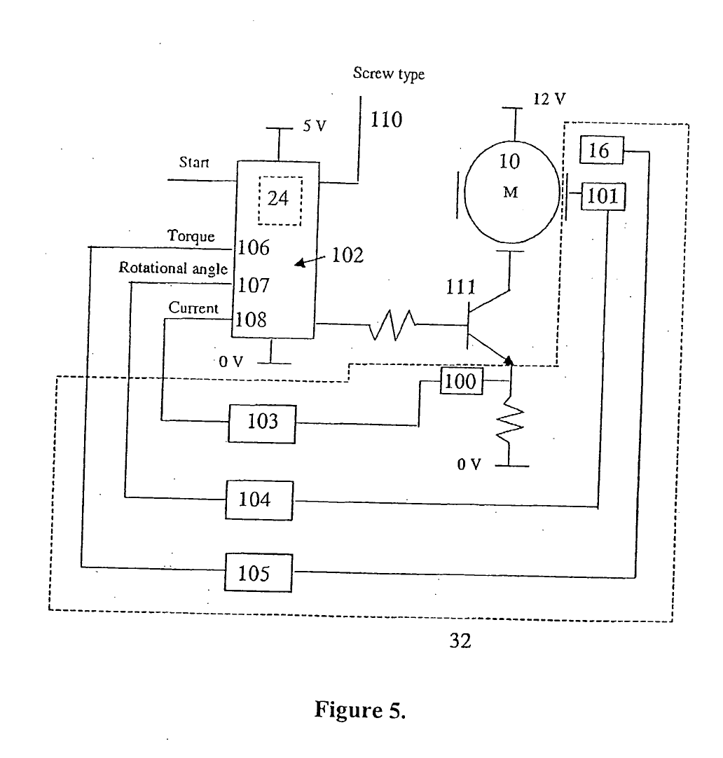

An adaptive apparatus for driving a threaded device 14 into host material such as biological tissue (in particular bone material 12), in accordance with a preferred embodiment of the present invention is shown in FIGS. 3, 4 and 5 of the accompanying drawings. The adaptive apparatus comprises a motorised drill/driver 10 having sensors in the form of strain-gauge instrumented torque transducers 16 for detecting the amount of torque exerted by the drill/driver 10 on the bone material 12 (and thus the amount of torque exerted by the bone material 12 on the drill/driver 10). In an alternative embodiment, the drill/driver has sensors in the form of motor current sensors 100. The drill/driver 10 is also provided with sensors in the form of additional transducers 101 for detecting a rotational-angle of a removable drilling bit 20 or driving bit 22 connected to the drill/driver via a chuck 26.

The values of torque and rotational angle, sensed by sensors 16 and 101, respectively, are processed using signal conditioning units 105 and 104 respectively. Conditioned signals representing torque 106 and rotational angle 107 are input to a microcontroller 102, and stored by memory means in the form of readable/writeable memory 24 in the microcontroller 102. These conditioned signals 106 and 107 are processed by the microcontroller 102 in comparison to known experimental reference data or algorithms stored in the microcontroller 102 in order to characterise the bone material 12 in terms of qualities related to its functional density, such as qualities of stiffness and strength. The known experimental data is in the form of quantitative relationships between, or algorithms relating, rotational angle, linear displacement, current, torque and screw type, and the comparison is done electronically using the microcontroller 102 with suitable software. The known experimental data or algorithms may be updated by replacing the software loaded into the microcontroller 102, or by uploading updated data or algorithms used by the software.

In a practical form of the invention, the signal conditioning units 105 may be in the form of a specific component known as an Analog Devices 1B31, and the microcontroller 102 may be in the form of a Microchip PIC18F452.

The approach of contact of the head 15 of the threaded device 14 with the surface of the bone material 12 is determined by the microcontroller 102 from the conditioned rotational angle 107 and screw type 110 data. Contact of the head 15 of the threaded device 14 on the bone material 12 is automatically detected by measuring the change in gradient of the torque curve using the microcontroller 102, as illustrated by “HC” (referring to Head Contact) in the examples shown in FIGS. 7, 11 and 15. The processing is performed continuously, and the characterising of the bone material 12 is continuously updated. The characterising of the bone material 12 is used to determine a shut-off condition at which safe and effective engagement of the threaded device 14 in the particular bone material 12 is achieved, the shut-off condition being in the form of a torque threshold value. Alternatively, the shut-off condition may be in the form of a rotational angle threshold value, or a relationship between torque and rotational angle. A feedback arrangement 32 is used to process the values of torque and rotational angle by way of the microprocessor 102 to calculate a dynamic torque value and a dynamic first derivative of torque with respect to rotational angle, and to compare this dynamic torque value and dynamic first derivative with the shut-off criterion and a head contact criterion, respectively. When the shut-off condition is achieved, a control system in the form of electronic circuitry containing a semiconductor switch 111 (for example a Motorola 2N3055A component) shuts off the power supply from the battery pack 36 to the drill/driver 10 to prevent over-tightening of the threaded device 14 within the bone material 12.

The above preferred embodiment has been described by way of example only and modifications are possible within the scope of the invention. In one particular alternative embodiment, the sensed quantity is motor current 103 detected by a current sensor 100. The current signal is used in the same way as the torque signal described in the above-described embodiment. Contact of the head 15 of the threaded device 14 on the bone material 12 may be automatically detected by measuring the change in gradient of the current curve using the microcontroller 102, as illustrated by “HC” (referring to Head Contact) in the examples shown in FIGS. 9, 13 and 17.

In yet another alternative embodiment, the driving device is pneumatic, the sensed quantity is or is related to pressure, and the shutoff condition is applied to a pneumatic regulator.

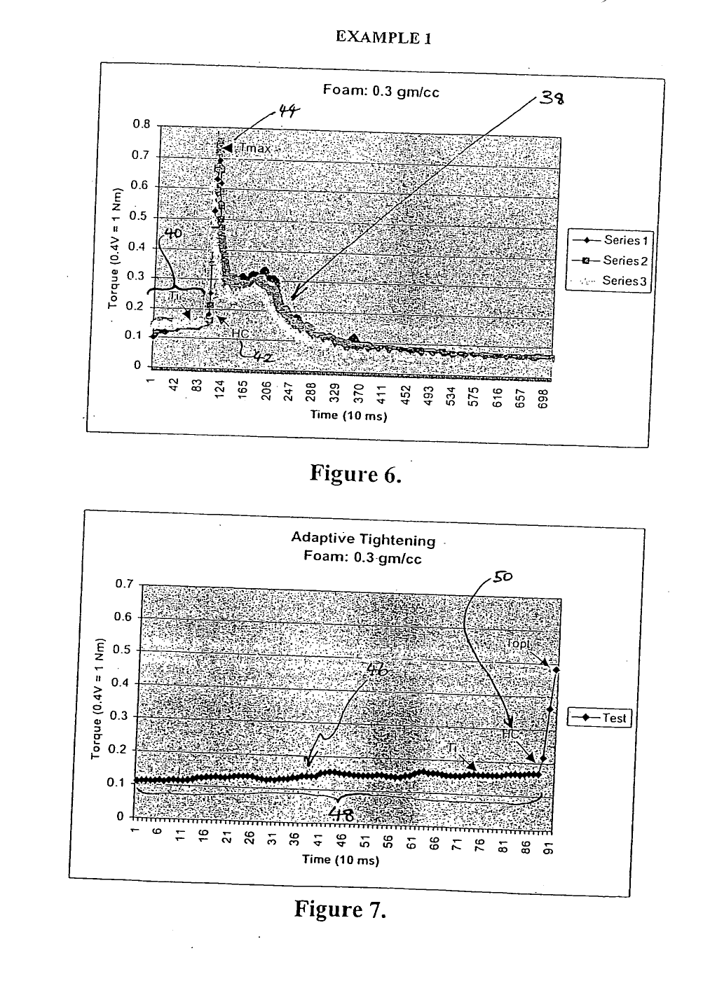

Although in the above-described preferred embodiment the host material is bone material, it should be noted that the host material may take other forms, such as the polyurethane foam as used in the experiments conducted to obtain the data represented in FIGS. 6 to 13.

### OPERATIONAL EXAMPLES

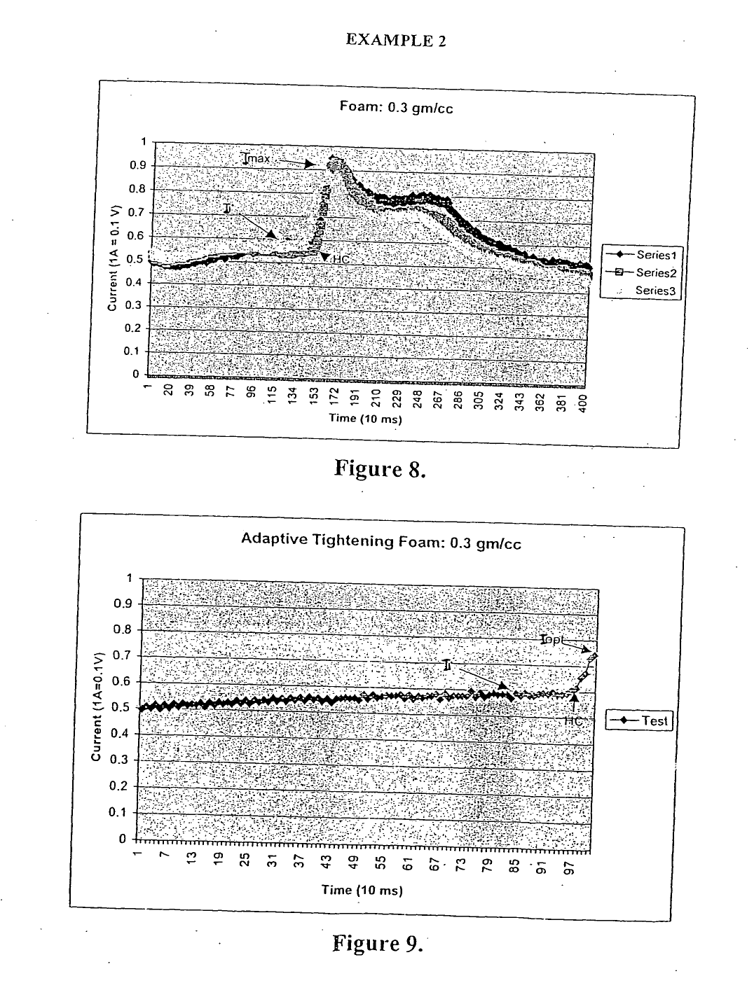

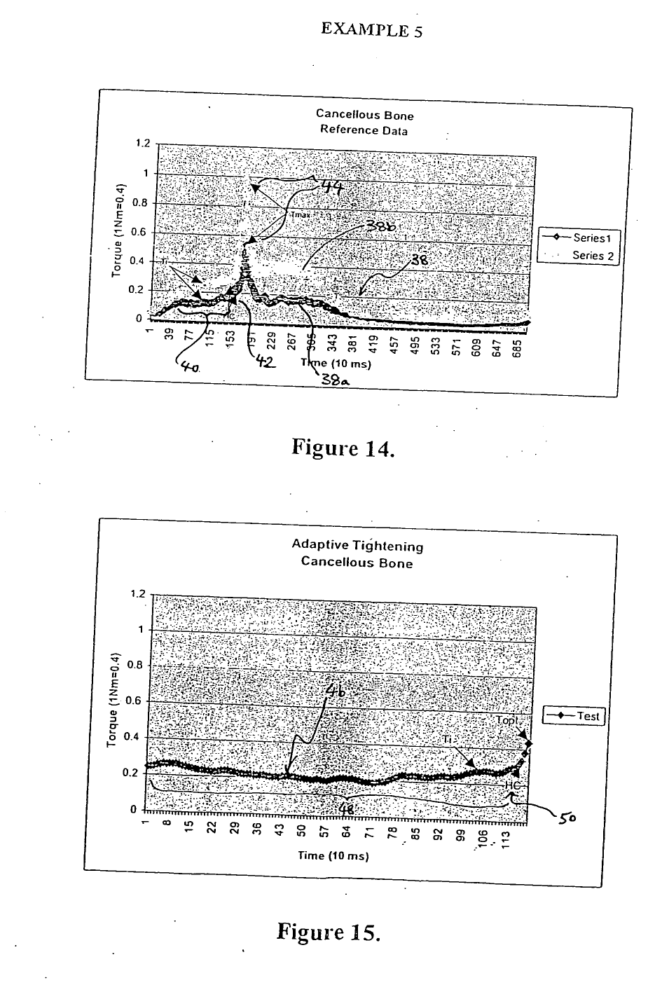

The process of adaptive tightening is illustrated with reference to Examples 1-6 shown in FIGS. 6-17. FIGS. 6, 7, 10, 11, 14 and 15 show signals from a torque transducer in relation to time, as a threaded device is driven into host material, over three different experimental threaded device/host material combinations. FIGS. 8, 9, 12, 13, 16 and 17 illustrate the same experimental combinations using current rather than torque. Six materials have been tested: polyurethane foam of two densities (0.2 and 0.3 gm/cc), cancellous bone (0.9 gm/cc), cortical bone (2 gm/cc), balsawood and meranti. The results from laboratory tests of three of these materials are shown in the Figures: polyurethane foam of two densities (0.2 and 0.3 gm/cc—see FIGS. 6, 7, 8, 9 and 10, 11, 12, 13 respectively, and cancellous bone (0.9 gm/cc)—see FIGS. 14, 15, 16, 17.

Stored Reference Data

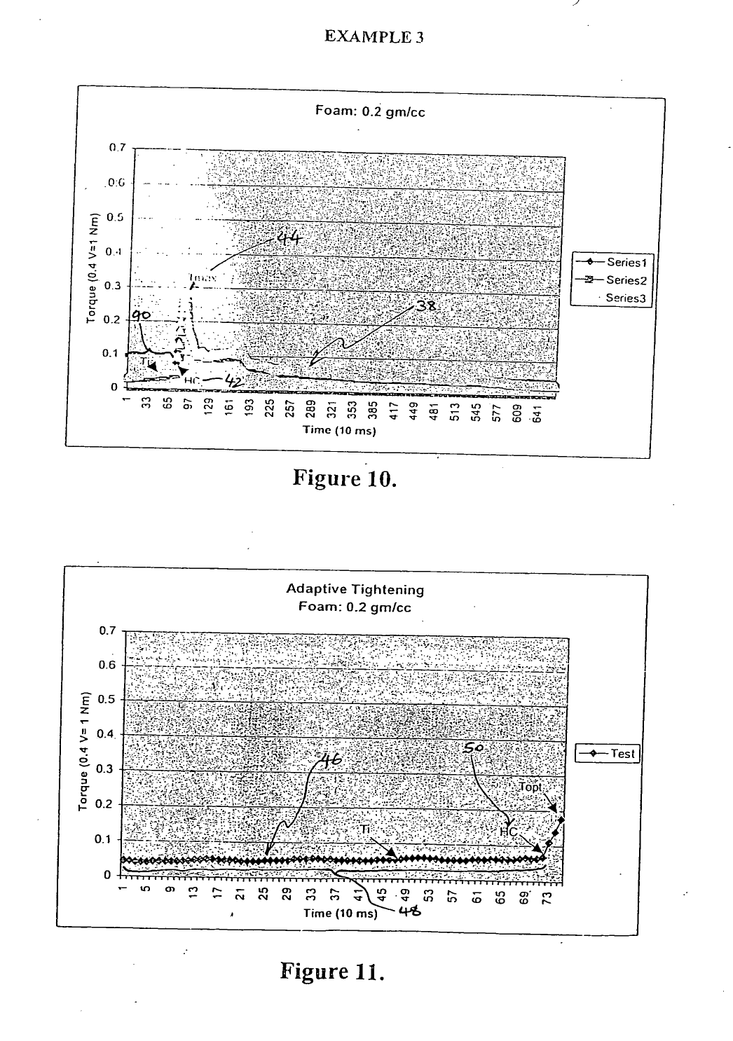

FIGS. 6 and 10 show examples of the known torque reference data stored on the microprocessor 102 for polyurethane foam of two densities: 0.2 and 0.3 gm/cc. The data from three trials of overtightening a 7.0 mm cancellous screw to failure of the host material is shown for each of the two densities. The torque curves 38 have characteristic features: (i) Ti, torque values in an initial region 40 of the torque curve 38 prior to contact of the head 15 of the threaded device 14 on the surface of the host material 12; (ii) HC (reference numeral 42), a sharp steepening of the torque curve 38 corresponding to engagement of the head 15 of the threaded device 14 with the surface of the host material 12; and (iii) Tmax (reference numeral 44), a peak torque value corresponding to failure of the sample host material 12 around the threaded device 14 thread. For the denser foam material (see FIG. 6), Ti values ranging between 0.15 and 0.18 correspond to Tmax values between 0.6 and 0.75. For the less dense material (see FIG. 10), Ti values between 0.03 and 0.06 correspond to Tmax values between 0.28 and 0.3.

Similarly, FIG. 14 shows an example of known torque reference data stored on the microprocessor 102 for cancellous bone of density 0.9 gm/cc, having Ti values ranging between approximately 0.1-0.3, and Tmax values of 0.5 and 1 for the two curves 38a,38b shown.

Adaptive Procedure

FIGS. 7, 11 and 15 illustrate the actual adaptive tightening in the three materials from FIGS. 6, 10 and 14, using the data represented in FIGS. 6, 10 and 14 as reference data stored in the microprocessor 102. The rotation signal together with the screw pitch identifies the region Ti prior to head contact. Processed torque values taken during driving of the threaded device 14 into the host material 12, represented by plots 46, are compared with the stored reference torque data stored in the microprocessor represented by torque curves 38 of FIGS. 6, 10 and 14. Torque values Ti, taken in the initial region of torque measurement 48 prior to contact of the head 15 of the threaded device 14 on the surface of the host material 12 of approximately 0.17 in FIG. 7 identify the material used as being 0.3 gm/cc foam by comparison with the torque reference data stored on the microprocessor 102, and in particular by comparison with the torque reference data represented by the torque curves 38 of FIG. 6. Once the material is characterised in this way, a safe shutoff condition in the form of a shutoff torque threshold is determined by using the known torque reference data for this material (ie. the data shown in FIG. 6 in the present example) by calculating the interval corresponding to between ⅓ and ⅔ of the range between Ti and Tmax of FIG. 6, and this shutoff condition is targeted. It should be noted that the values of ⅓ and ⅔ are arbitrary safety factors, and could conceivably be other values between 0 and 1. In this particular example, the shutoff torque threshold for 0.3 gm/cc foam (with reference to the values of Ti and Tmax obtained from FIG. 6) corresponds to a range of processed values of torque of approximately 0.4 to 0.6. As can be seen in FIG. 7, the final value of torque obtained Topt is approximately 0.5, and is within the shutoff torque threshold range calculated.

Software stored on the microprocessor 102 continuously calculates the dynamic slope of the processed torque values, as represented by plot 46, with respect to time, by numerical differentiation. A rapid increase in slope identifies contact of the head 15 of the threaded device 14 with the surface of the material 12, ie. head contact (HC) as indicated by reference numeral 50. This activates a condition of the system wherein the shutoff torque threshold has been determined and is sensed for until achieved.

The process is similarly illustrated for the lower density 0.2 gm/cc foam material in FIGS. 10 and 11, and for 0.9 gm/cc cancellous bone in FIGS. 14 and 15. In the case of 0.2 gm/cc foam, in FIG. 11, values of Ti around 0.06 fall within the Ti range for 0.2 gm/cc material, as established in the known data shown in FIG. 10. A shutoff torque threshold range between 0.15 and 0.25 is then identified with reference to the stored information, by determining the interval between ⅓ and ⅔ of the range between Ti and Tmax of FIG. 10. Head contact is detected by the change in slope of the torque plot 46, as before. The motor is then shut off once an optimum torque value within the shutoff torque threshold range has been reached for this material. In the example in FIG. 11, Topt is 0.2.

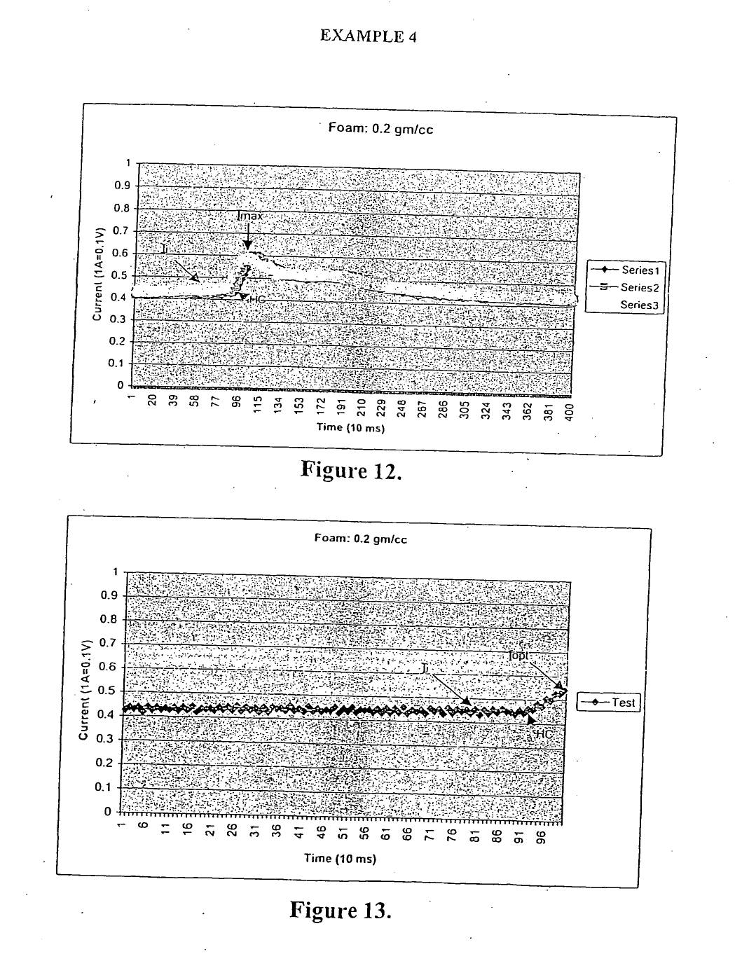

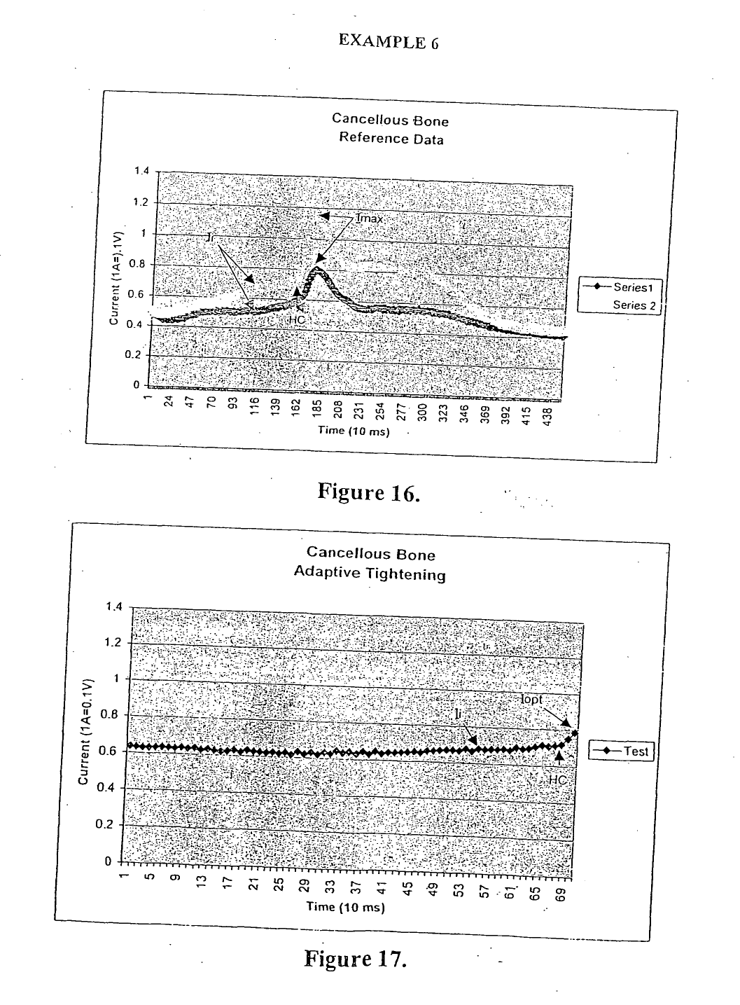

The same general procedure is followed when the sensed quantity is current, as shown in FIGS. 8, 9 (for 0.3 gm/cc foam), FIGS. 12, 13 (for 0.2 gm/cc foam), and FIGS. 16, 17 (for 0.9 gm/cc cancellous bone). Stored reference data and the corresponding adaptive procedure using current are demonstrated for the same three materials in these Figures.

In an alternative embodiment, the first sensed quantity is torque and the second sensed quantity is current. In this embodiment, the same general procedure as described above is followed, and both torque and current data may be used to characterise the host material, to determine the shut-off condition, and to control ceasing of rotation of the driving bit.

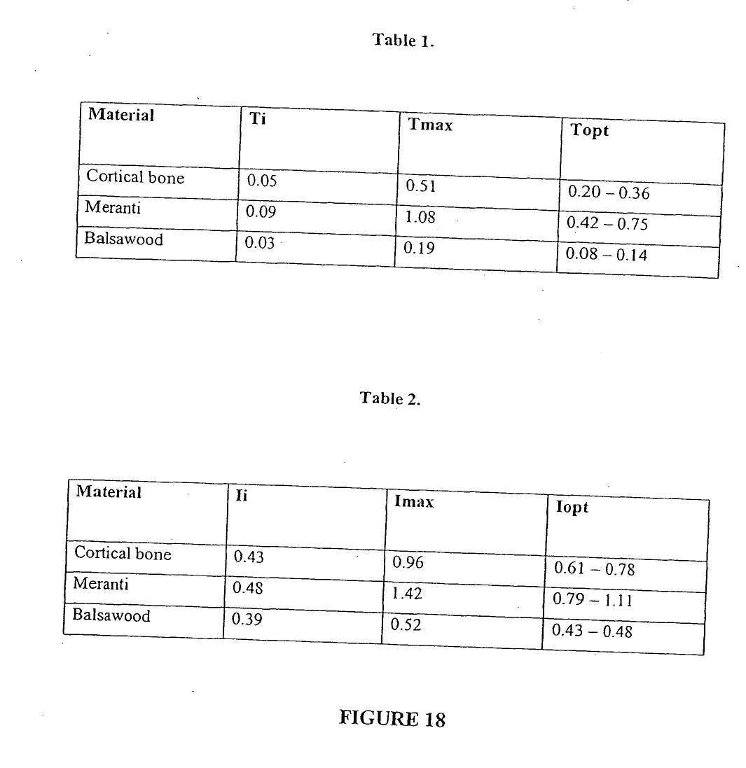

The results of further laboratory measurement in host materials of cortical bone, hardwood (meranti) and balsawood, using torque and current, are summarised in FIG. 18 in Tables 1 and 2, respectively. In particular, Table 1 shows examples of output voltage of a torque transducer (0.4 V=1 Nm) for initial level prior to head contact (Ti), maximum value corresponding to thread stripping (Tmax) and corresponding optimal adaptive range of tightening (Topt), for cortical bone (ovine tibia), hardwood (meranti) and balsawood. Table 2 shows examples of output voltage corresponding to motor current (0.1 V=1 A) for initial level prior to head contact (Ii), maximum value corresponding to thread stripping (Imax) and corresponding optimal adaptive range of tightening (Iopt), for cortical bone (ovine tibia), hardwood (meranti) and balsawood.

The preferred embodiments and the above operational examples have been described by way of example only and modifications are possible within the scope of the invention.

1. An adaptive apparatus for driving a threaded device into material, the apparatus including a rotating driving bit, a sensor for sensing a first quantity related to the material's properties, a memory storing sensed values of said first quantity, and a feedback arrangement which processes the values sensed by the sensor in comparison to known data to characterize the material and to determine a shut-off condition at which safe and effective engagement of the threaded device in the material is achieved, wherein the feedback arrangement ceases rotation of said driving bit when said shut-off condition is achieved.

2. An adaptive apparatus as claimed in claim 1, wherein the processing of the values sensed by the sensor is performed continuously.

3. An adaptive apparatus as claimed in claim 1, wherein contact between a head of the threaded device and the material is automatically detected by the apparatus, and initial values of said first quantity are sensed prior to said contact.

4. An adaptive apparatus as claimed in claim 3, wherein said contact between the head of the threaded device and the material is detected by sensing said first quantity.

5. An adaptive apparatus as claimed in claim 1, wherein the apparatus includes a drill bit for drilling the material so that said threaded device can be driven into a hole fanned by said drill bit.

6. An adaptive apparatus as claimed in claim 5, wherein the first-mentioned sensor senses said first quantity during said drilling to supplement values of said first quantity sensed during driving, and subsequent values of said first quantity sensed during said drilling are stored in said storage means during said drilling, and said feedback arrangement processes these values in comparison to known data to characterize the material and to determine said shut-off condition at which safe and effective engagement of the threaded device in the material is achieved.

7. An adaptive apparatus as claimed in claim 1 wherein the apparatus includes a further sensor for sensing a second quantity, and subsequent values of said second quantity are stored in said memory means and are processed in combination with values of said first quantity and in comparison to known data to characterize the material and to determine said shut-off condition.

8. An adaptive apparatus as claimed in claim 7, wherein the second quantity is angular rotation of either the drilling or driving bit.

9. An adaptive apparatus as claimed in claim 7, wherein the second quantity is axial displacement of the threaded device in the material.

10. An adaptive apparatus as claimed in claim 1, wherein the rotating driving bit is powered by a motor, and the feedback arrangement processes the quantity or quantities sensed by the one or more sensors to control rotation of the motor to prevent over-tightening.

11. An adaptive apparatus as claimed in claim 1, wherein said first quantity is or is related to an amount of resistive torque exerted by the material on the threaded device.

12. An adaptive apparatus as claimed in claim 1, wherein said first quantity is current of a motor of the adaptive apparatus.

13. An adaptive apparatus as claimed in claim 1, wherein the rotating driving bit is driven pneumatically, said first quantity is or is related to pressure, and control is effected by means of a regulator.

14. An adaptive apparatus as claimed in claim 1, wherein the first quantity is axial force on the drilling or driving bit.

15. An adaptive apparatus as claimed in claim 1, wherein the apparatus is self-contained and is portable in a hand-held form.

16. An adaptive apparatus as claimed in claim 1, wherein the threaded device is a screw.

17. An adaptive apparatus as claimed in claim 1, wherein the feedback arrangement characterizes the material by comparing the magnitude of values of said first quantity, sensed prior to contact of the head of the threaded device on the surface of the material, to known data for different material types to isolate a relevant set of known data, and the shut-off condition is determined by ascertaining an expected failure threshold from the relevant set of known data and by applying a safety factor to the expected failure threshold.

18. A method of driving a threaded device into material, said method including the steps of:
driving the threaded device into said material by way of a rotating driving bit;
sensing a first quantity related to material properties of the material;
storing sensed values of said first quantity;
processing the values sensed by the sensor in comparison to known data to characterize the material and to determine a shut-off condition at which safe and effective engagement of the threaded device in the material is achieved; and
ceasing rotation of said driving bit when said shut-off condition is achieved.

19. A method of driving a threaded device as claimed in claim 18, wherein the processing step is performed continuously.

20. A method of driving a threaded device as claimed in claim 18, wherein said method further includes the steps of:
automatically detecting contact between a head of the threaded device and the surface of the material; and
sensing initial values of said first quantity prior to said contact.

21. A method of driving a threaded device as claimed in claim 18, wherein said method further includes the step of automatically detecting contact between the head of the threaded device and the material by sensing said first quantity.

22. A method of driving a threaded device as claimed in claim 18, wherein said method further includes the step of drilling said material so that said threaded device can be driven into a hole formed by said drilling step.

23. A method of driving a threaded device as claimed in claim 22, wherein said method further includes the steps of:
sensing subsequent values of said first quantity during said drilling to supplement values of said first quantity sensed during driving;
storing these sensed values; and
processing the values sensed during the drilling step in comparison to known data to characterise the material and to determine said shut-off condition at which safe and effective engagement of the threaded device in the material is achieved.

24. A method of driving a threaded device as claimed in claim 18, wherein said method further includes the steps of:
sensing a second quantity;
storing subsequent values of said second quantity; and
processing values of said second quantity in combination with values of said first quantity and in comparison to known data to characterize the material and to determine said shut-off condition.

25. A method of driving a threaded device as claimed in claim 24, wherein the second quantity is angular rotation of the driving bit.

26. A method of driving a threaded device as claimed in claim 24, wherein the second quantity is axial displacement of the threaded device in the material.

27. A method of driving a threaded device as claimed in claim 18, wherein said first quantity is or is related to an amount of resistive torque exerted by the material on the threaded device.

28. A method of driving a threaded device as claimed in claim 18, wherein said first quantity is current of an electric motor operating the rotating driving bit.

29. A method of driving a threaded device as claimed in claim 18, wherein the first quantity is axial force exerted by the material on the driving bit.

30. A method of driving a threaded device as claimed in claim 18, wherein, the rotating driving bit is driven pneumatically, said first quantity is or is related to pressure, and control is effected by means of a regulator.

31. A method of driving a threaded device as claimed in claim 18, wherein the method further includes the steps of:
characterizing the material by comparing the magnitude of values of said first quantity sensed prior to contact of the head of the threaded device on the surface of the material to known data for different material types to isolate a relevant set of known data, and
determining the shut-off condition by ascertaining an expected failure threshold from the relevant set of known data and by applying a safety factor to the expected failure threshold.

32. An adaptive apparatus as claimed in claim 1 wherein the material is bone.

33. An adaptive apparatus as claimed in claim 1 wherein the material is tooth material.

34. An adaptive apparatus as claimed in claim 1 wherein the material is wood.

35. An adaptive apparatus as claimed in claim 1 wherein the material is synthetic.

36. A method of driving a threaded device as claimed in claim 18, wherein the material is bone.

37. A method of driving a threaded device as claimed in claim 18, wherein the material is tooth material.

38. A method of driving a threaded device as claimed in claim 18, wherein the material is wood.

39. A method of driving a threaded device as claimed in claim 18, wherein the material is synthetic.

40. An adaptive apparatus for driving a threaded device relative to a body of material, the apparatus including a rotating driving bit, a sensor for sensing a first quantity related to a material property of the body, a memory storing sensed values of said first quantity, and a feedback arrangement which processes the values sensed by the sensor in comparison to known data to characterize the material of the body and to determine a shut-off condition at which safe and effective engagement of the threaded device to the material is achieved, wherein the feedback arrangement ceases rotation of said driving bit when said shut-off condition is achieved.

41. A method of driving a threaded device relative to a body of material, said method including the steps of:
driving the threaded device relative to said material by way of a rotating driving bit;
sensing a first quantity related to a material property of the body;
storing sensed values of said first quantity;
processing the values sensed by the sensor in comparison to known data to characterise the material of the body and to determine a shut-off condition at which safe and effective engagement of the threaded device to the material is achieved; and
ceasing rotation of said driving bit when said shut-off condition is achieved.
